(19) United States
(12) Patent Application Publication
(10) Pub. No.: US 2002/0169300 A1
(43) Pub. Date: Nov. 14, 2002

(54) Method of detection and treatment of colon cancer by analysis of beta-catenin-sensitive isoforms of lymphoid enhancer factor-1

(76) Inventors: Marian L. Waterman, Irvine, CA (US); Randall F. Holcombe, Cotode Caza, CA (US); J. Lawrence Marsh, Newport Beach, CA (US); Karine Hovanes, Westminster, CA (US); Tony Wai Hung Li, Los Angeles, CA (US)

Correspondence Address: Lisa A. Haile, Ph.D., Gray Cary Ware & Freidenrich LLP, Suite 1100, 4365 Executive Drive, San Diego, CA, 92121-2133, US

(21) Appl. No.: 10/060,844

(22) Filed: Jan. 29, 2002

Related U.S. Application Data
(60) Provisional application No. 60/265,264, filed on Jan. 30, 2001.

(51) Int. Cl. C07H 21/02; C07H 21/04; C07K 1/00; C07K 14/00; C07K 17/00

(52) U.S. Cl. 536/23.1; 530/350

(57) ABSTRACT

The present invention relates to the discovery that LEF1 is a new type of target gene in that it is ectopically activated in colon cancer. The pattern of this ectopic expression is unusual because it derives from selective activation of a promoter for a full-length LEF1 isoform that binds β-catenin, but not a second, intronic promoter that drives expression of a dominant negative isoform. β-catenin/TCF complexes can activate the promoter for full-length LEF1 suggesting that in cancer, high levels of these complexes misregulate transcription to favor a positive feedback loop for Wnt signaling by inducing selective expression of full length, β-catenin sensitive forms of LEF/TCFs. The invention provides diagnostic and therapeutic methodologies based on the discoveries described herein.

This application claims priority under 35 U.S.C. §119(e) to U.S. Provisional Patent Application Ser. No. 60/265,264, filed Jan. 30, 2001, the entire contents of which is incorporated herein by reference.

This invention was made in part with government support under Grant No. HD3608 1, HD36049 and CA-83982 awarded by the National Institutes of Health (NIH). The government may have certain rights in this invention.

### BACKGROUND OF THE INVENTION

1. Field of the Invention

The invention relates generally to cancer diagnostics and therapeutics and, more specifically, to aberrant activation and expression of lymphoid enhancer factor (LEF1) in colon cancer.

2. Background Information

Constitutive activation of the Wnt signaling pathway is a root cause of many colon cancers¹⁻³. Activation of the pathway is caused by genetic mutations that stabilize the β-catenin protein, allowing it to accumulate in the nucleus and form complexes with any of the four members of the lymphoid enhancer factor (LEF1) and T-cell factor (TCF1, TCF3, TCF4) family of transcription factors (referred to collectively as LEF/TCFs) to activate transcription of target genes^{3, 4}. Target genes such as MYC, CCND1, MMP-7, and TCF7 (refs. 5-9) are normally expressed in colon tissue, so it is proposed that abnormal expression levels or patterns imposed by β-catenin/TCF complexes play a role in tumor progression.

### SUMMARY OF THE INVENTION

The present invention relates to the seminal discovery that LEF1 is a new type of target gene in that it is ectopically activated in colon cancer. The pattern of this ectopic expression is unusual because it derives from selective activation of a promoter for a full-length LEF1 isoform that binds β-catenin, but not a second, intronic promoter that drives expression of a dominant negative isoform. β-catenin/TCF complexes can activate the promoter for full-length LEF1 suggesting that in cancer, high levels of these complexes misregulate transcription to favor a positive feedback loop for Wnt signaling by inducing selective expression of full length, β-catenin sensitive forms of LEF/TCFs.

In one embodiment, the invention provides an isolated polynucleotide comprising a truncated LEF1 polynucleotide or homolog thereof lacking nucleotides encoding a J-catenin binding domain and not adjacent to nucleotide sequences to which it is naturally adjacent. In a particular aspect, the polynucleotide encodes a polypeptide having about 283 amino acids beginning at a methionine codon within exon 3 of the human LEF-1 gene. In another aspect, the polynucleotide encodes a polypeptide beginning at about amino acid residue 116 of human LEF-1.

The invention also includes an isolated polynucleotide having regulatory activity and comprising nucleotides in intron 2 of human LEF-1 gene and within about 50 nucleotides 5′ of the third exon of human LEF-1 gene and homologs thereof (e.g., other species such as ovine, bovine, avian, murine, etc.).

The invention also includes a purified polypeptide encoded by a polynucleotide described herein (e.g., a dominant negative truncated LEF-1 protein). In another embodiment, the invention includes isolated antibodies that bind specifically to a polypeptide encoded by a polynucleotide of claim 1, or to immunogenic fragments thereof, with the proviso that the antibodies do not bind to human LEF-1 polypeptide or immunogenic fragments thereof. The antibodies may be polyclonal or monoclonal.

In addition, the invention includes a second downstream promoter of LEF-1, described in the examples, operably linked to either a polynucleotide described herein, or a polynucleotide of interest (e.g., a polynucleotide encoding a therapeutic protein or a therapeutic polynucleotide such as an antisense molecule).

In yet another embodiment, the invention provides a method for diagnosing or monitoring the recurrance or predisposition to colon cancer in a subject comprising detecting the level of expression of full length LEF-1 and truncated LEF-1 polynucleotide or the level of full length LEF-1 and truncated LEF-1 polypeptide in a sample from the subject, wherein an elevated level of fall length LEF-1 polynucleotide or polypeptide is indicative of the presence of colon cancer or predisposition thereto. In a preferred embodiment, the subject is a human.

The invention also provides a kit useful for for diagnosing or monitoring the recurrance or predisposition to colon cancer in a subject comprising a first container containing a nucleic acid probe for detecting the level of expression of full length LEF-1 and truncated LEF-1 polynucleotide. In another embodiment, the invention provides a kit useful for for diagnosing or monitoring the recurrance or predisposition to colon cancer in a subject comprising a first container containing an antibody for detecting the level of expression of full length LEF-1 and truncated LEF-1 polypeptide.

The invention also provides an isolated polypeptide comprising a LEF1 amino acid sequence consisting of a C-terminal fragment of LEF1, wherein the C-terminal fragment is from amino acid 116 to 398 of LEF1 and wherein the LEF1 amino acid sequence is not adjacent to an amino acid sequence that is naturally adjacent to the LEF1 amino acid sequence.

The invention provides a method of treating or inhibiting colon cancer in a subject comprising contacting a cell with an antagonist of a regulatory region encoding full length LEF-1 or an agonist (e.g., small molecule) of the polynucleotide of the invention, thereby treating or inhibiting colon cancer.

In yet another embodiment, the invention provides a method of treating or inhibiting colon cancer in a subject comprising contacting a cell with an antagonist of a full length LEF-1 polypeptide or an agonist of a truncated LEF-1 polypeptide, thereby treating or inhibiting colon cancer.

In another embodiment, the invention provides a method for screening for an agent (e.g., a compound, small molecule, peptide, mimetic, antisense, etc.) useful for the treatment of colon cancer comprising contacting a promoter sequence of LEF-1 or a truncated LEF-1 promoter operably associated with a detectable marker with a test agent, and detecting a decrease in detectable marker from the promoter of LEF-1 or an increase in detectable marker from the truncated promoter is indicative of an agent that is useful for the treatment of colon cancer.

### BRIEF DESCRIPTION OF THE DRAWINGS

FIG. 1 shows LEF1 and TCF7L2 (gene for TCF4 protein) expression in normal human colon tissue and human colon carcinomas. In situ hybridization with digoxigenin-labeled sense and antisense RNA complementary to the 3′ untranslated regions of human LEF1 or TCF7L2 mRNA were used as probes to detect endogenous messages in colon tissue. LEF1 mRNA (a-e), is not expressed whereas TCF7L2 mRNA (f-j), is highly expressed in normal colon tissue. Both LEF1 and TCF7L2 expression are detected in human colon carcinomas. Expression was detected in 10 of 10 colon carcinoma samples each derived from separate patients (k-o). Detection is specific as antisense, but not sense RNA probes (inset) detect high level expression of LEF1 and TCF7L2 in Jurkat T cells (p, q). Magnification for a, f is 10×(size bar is 100 mm), for b, d, g, i is 40×(size bar is 10 mm), and for c, e, h, j, k-q is 100×(size bar is 10 mm).

FIG. 2 shows a Northern analysis of LEF1 expression in normal thymus tissue and cancer cell lines and identification of a second promoter in intron 2 of human LEF1. a, Total RNA or polyA+RNA from the indicated tissues and cell lines was analyzed with probes from two different regions of the LEF1 cDNA. A probe from the open reading frame (ORF) detects two mRNAs of 3.6 kb and 2.2 kb. A probe from the 5′ untranslated region detects only the 3.6 kb mRNA (5′ UTR; the total length of the 5′ UTR in exon 1 is 1,186 nucleotides). Jurkat and 2017 cells 21 are human and mouse T lymphocyte cell lines respectively and Colo 320, DLD1, Colo 205 cells are derived from human colon carcinomas. Murine RNAs are not detected with the 5′ UTR probe because the nucleotide sequence in this region diverges significantly between human and mouse (Hovanes and Waterman, data not shown). The same Northern blot was probed with a control probe (GAPDH). b, LEF1 contains a promoter in intron 2. Fragments from the second intron of LEF1 were tested for promoter activity in Jurkat T lymphocytes using the pGL2 luciferase reporter plasmid. A 232 nucleotide fragment (EspI-XhoI) can act as a promoter for transcription in the forward but not the reverse orientation. Luciferase light units varied from 500 to 15,000. Data are derived from duplicate samples, and the results shown represent one of four replicative experiments. Fold activation was calculated as a ratio of luciferase levels from each reporter construct relative to the promoter-less pGL2 plasmid (vector). A schematic of exons 1-3 shows the relative positions of the introns, promoters and coding sequences for the LEF 1 bcatenin binding domain.

FIG. 3 shows LEF1 produces two different protein products that differ at the N-terminus. a, Predicted LEF1 protein products from the 3.6 and 2.2 kb mRNAs. The shorter LEF1 protein begins at amino acid 116 within the full length LEF1 sequence and is missing the b-catenin binding domain and a portion of the context-dependent activation domain. b, Jurkat T lymphocytes, but not colon cancer cells express LEF 1DN. Whole cell extracts (50,000 cell equivalents) were analyzed on western blots probed with monoclonal antibodies specific for fulllength LEF1 (REMB 1, Exalpha Biologicals), and TCF4 or TCF1 proteins (Upstate Biotech). REMB6 (Exalpha Biologicals) is a monoclonal antibody raised against LEF1 protein, but recognizes an epitope in the HMG box that is highly conserved in LEF/TCF family members. Polyclonal LEF1 antisera recognizes conserved epitopes in all mammalian LEF/TCF family members and isoforms. TCF polypeptides that cross-react with REMB6 and LEF 1 polyclona antibody are indicated by *. A polypeptide of 38 kD (LEF1 DN; asterisk) is detected by the LEF1 polyclonal antisera and REMB6 but not REMB1 and therefore matches the predicted structure of LEF1DN. A 2.2 kb in vitro transcribed RNA produces a single 38 kD LEF1DN product in rabbit reticulocyte lysates. Lanes 1-3 contain whole cell lysates from Jurkat T lymphocytes as a reference for the whole cell lysate from normal human peripheral blood lymphocytes in lane 4. Full-length LEF1 polypeptides are indicated in whole cell extracts from Jurkat cells and the colon cancer cells SW480 and Colo320 (75,000 cell equivalents). LEF1DN is only detected in Jurkat extract and is not present in the extracts from colon cells. c, LEF1DN can repress activation of reporter gene expression by β-catenin. The LEF/TCF reporter plasmid TOPtk was co-transfected into 2017 T lymphocytes with increasing amounts of an expression vector for DNLEF, a truncated form of LEF1 similar in structure to LEF1DN (aa 67-399) (ref. 22). Endogenous LEF/TCFs in Jurkat cells are able to work with b-catenin to activate the reporter gene 15-fold, but in the presence of DNLEF1, activation is reduced to basal levels.

FIG. 4 shows LEF-b 1DN can repress activation of reporter gene expression by β-catenin. The LEF/TCF reporter plasmid TOPtk was co-transfected into Jurkat T lymphocytes with increasing amounts of an expression vector for ΔNLEF (amounts are indicated in micrograms of co-transfected plasmid), a truncated form of LEF1 similar in structure to LEF-1DN (aa67-399)The LEF1 promoter is activated by TCF1 and TCF4-β-catenin complexes in 2017 T lymphocytes. a, A luciferase reporter gene driven by the LEF1 promoter (−672, +305) was co-transfected with expression vectors for full length TCF1E or TCF4E and β-catenin. Activation was calculated using equivalent amounts of empty expression vector. TCF1E activated luciferase gene expression 7.0-fold and TCF4E activated 4.6-fold in this representative experiment. Fold activation by TCF1 over 5 replicate experiments is 8+3.65 (SD), for TCF4, 5.6-fold +3.7 (SD). Co-transfection of TCF1E and D19 β-catenin, a mutant that cannot bind to LEF/TCF proteins, did not activate the promoter. b, DNAase I footprint analysis of the LEF1 promoter with recombinant LEF1 protein reveals two binding sites downstream of the start site of transcription. The footprints are centered over two close matches to LEF/TCF consensus binding sites (YCTTTGWW): TCTTTGCTTT (+190) and TCTTTGTTC (+283). A fast migrating portion of intact probe obscures the +190 footprint with LEF1 protein in the second panel. Whole cell extracts from Jurkat T lymphocytes (express TCF4, TCF1 and LEF1) but not HeLa cells (little to no LEF/TCF expression) protect the +283 site but not the +190 site. c, Fragments of the LEF1 promoter were cloned into pGL2-enhancer plasmids and tested for activation by TCF1 and b-catenin. The region responsive to TCF/β-catenin encompasses the downstream LEF/TCF binding sites. Activation of the largest fragment (−672, +305) was 9.2-fold, whereas activation of fragments that delete the +283 LEF/TCF binding site with (−672, +262) or without (−64, +262) the upstream sequences are activated 4.3− and 3.6-fold respectively. Removal of both the +190 and +283 binding sites (to +78) reduces activation to 1.6-fold. d, Transient overexpression of a GFP/APC fusion protein in SW480 cells reduces LEF1 promoter reporter gene activity (−672, +305) three-fold. The parent construct which expresses only the GFP portion does not inhibit promoter activity. Whole cell extracts from Colo320 cells overexpressing GFP/APC were analyzed by western analysis with b-catenin monoclonal antisera, and LEF/TCF polyclonal antisera (75,000 cell equivalents; inset). A decrease in b-catenin and LEF1 levels is observed, but not a decrease in TCF4 levels (indicated by filled circle).

### DETAILED DESCRIPTION OF THE INVENTION

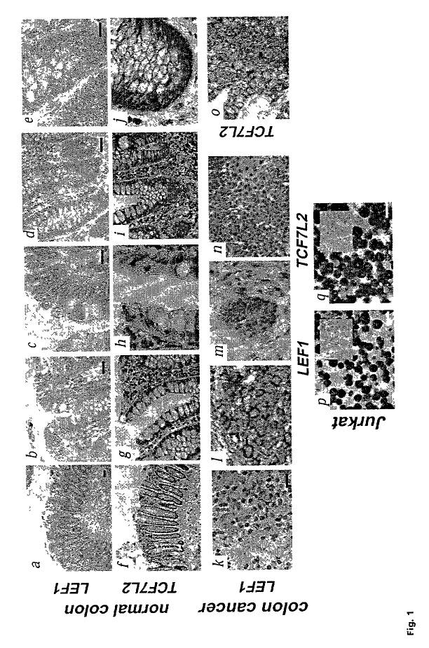

The present inventors have shown by at least Northern analysis that the LEF1 gene is often expressed in colon cancer cell lines whereas it is not detectable in normal colon tissue^{10, 11}. Here we used in situ hybridization to determine if LEF1 expression occurs in primary colon cancer tissue from patient biopsies and to determine if it is expressed in a small population of normal colon cells in crypts. Since the LEF/TCF family member TCF4 is expressed in normal colon11, we used human TCF4 probes as a reference. In striking contrast to TCF4, we did not detect LEF1 mRNA in normal mucosal tissue, not even in minor subpopulations of cells in the crypts of colon (FIG. 1 a-j). However, we detected LEF1 mRNA in all colon carcinoma biopsies analyzed (10 out of 10, FIG. 1k-n). We conclude that within the limits of detection for in situ hybridization, the LEF1 gene is not expressed in any cell in normal colon tissue but is aberrantly activated during colon carcinogenesis.

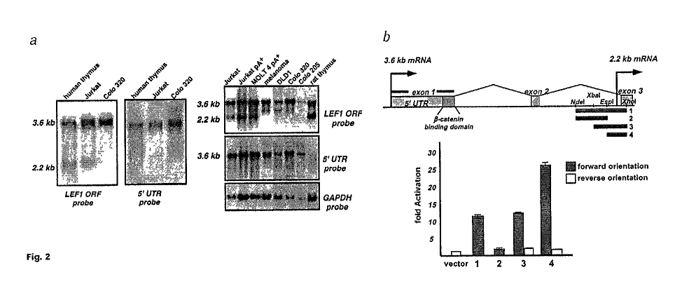

In normal thymus tissue, two mRNAs of 3.6 kb and 2.2 kb are produced from LEF1 (FIG. 2a, refs. 12, 13). However, in colon cancer and melanoma cells only the 3.6 kb mRNA is present (Colo 320, DLD1, Colo205, melanoma, and otherslo; FIG. 2a). Previously we determined that the 3.6 kb mRNA contains 1.2 kb 5′ and 3′ untranslated regions and a 1.2 kb open reading frame encoding a full length LEF1 polypeptide with β-catenin and HMG DNA binding domains 14. Here we probe the structure of the 2.2 kb mRNA by Northern analysis (FIG. 2a). Whereas probes from the LEF1 open reading frame and 3′ UTR could hybridize to both 3.6 and 2.2 kb mRNAs, we could not detect the 2.2 kb mRNA with a probe from exon 1 (5′ UTR, FIG. 2a). Extensive screening of cDNA libraries and other methods such as 5′ RACE did not uncover any evidence for alternative splicing to generate a smaller 2.2 kb mRNA (K. Hovanes, data not shown), therefore we considered the possibility of a second, downstream promoter.

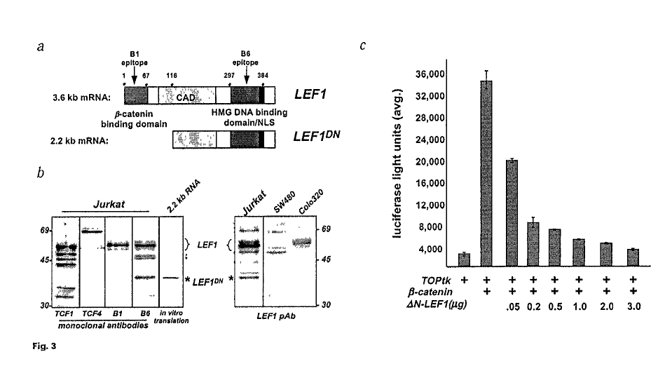

The exon and intron structure of human LEF1 and TCF1 are highly similar and both genes express similar sets of isoforms 14-16. Although TCF1 produces only one detected mRNA on Northern blots, a second promoter in intron 2 drives expression of an additional, similarly sized mRNA encoding a truncated TCF1 isoform that does not have the β-catenin binding domain¹⁵. We searched introns 1 and 2 of LEF1 for regions containing a promoter and detected activity with fragments of the second intron when they were cloned into a luciferase reporter vector in the forward but not the reverse direction (XbaI-XhoI, EspI-XhoI, FIG. 2b). Within the smallest of these fragments is a consensus TATA box motif 50 nucleotides 5′ of the third exon. Promoter activity is destroyed when we delete these 50 nucleotides (T. Li, data not shown). The predicted protein product from this second promoter is a 283 amino acid polypeptide beginning at a methionine codon within exon 3 (amino acid 116 within full length LEF1) and is thus missing the β-catenin binding domain and crucial amino acids in the context-dependent activation domain (CAD, FIG. 3a). We mapped the transcription start site within the second promoter (T. Li, data not shown), and a 2.2 kb RNA beginning at this +1 position and including all downstream exons was generated for in vitro translation. A single 38 kD polypeptide was produced in this reaction (asterisk, FIG. 3b). Using LEF1 polyclonal antisera for western analysis, we detected a 38 kD polypeptide in extracts from Jurkat T lymphocytes that express 3.6 kb and 2.2 kb LEF1 mRNAs but not in extracts of SW480 or Colo320 colon cancer cells that express only the 3.6 kb mRNA (LEF1 pAb, FIG. 3b). We also used LEF1, TCF1 and TCF4 specific monoclonal antibodies to confirm that this polypeptide is a product of LEF1 and contains the HMG DNA binding domain but not the b-catenin binding domain (REMB1, REMB6, TCF1, TCF4, FIG. 3b).

Overexpression of this truncated LEF1 isoform represses the ability of b-catenin to activate reporter gene expression (AN-LEF1, FIG. 3c). Repression must occur because the truncated LEF1 protein can bind to the LEF/TCF sites and prevent b-catenin recruitment to the target reporter plasmid. Therefore, the 38 kD LEF1 protein may function as a natural antagonist for Wnt signaling and hereafter shall be referred to as LEF1DN for “dominant negative”. The structure of LEF1DN is similar to a truncated TCF1 isoform that can function as a dominant negative to suppress activation of reporter genes by full length TCF proteins^{11, 17}. Expression of dominant negative forms of LEF/TCFs may be a general feature of LEF/TCF loci used to moderate the effects of Wnt signaling by competing with full length LEF/TCFs for target gene occupancy.

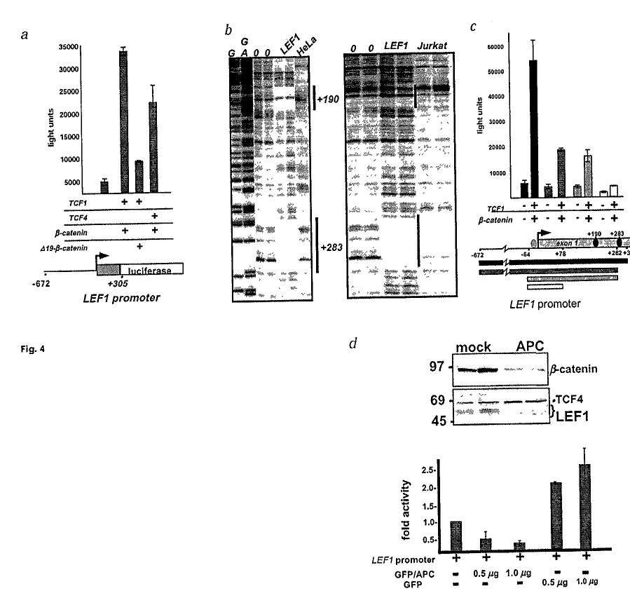

Since no LEF1 mRNA is detected in normal colon tissue, expression in cancer must be due to inappropriate activation of the first LEF1 promoter. We tested whether b-catenin/TCF complexes regulate the LEF1 promoter because it is known that Wnt3a can induce expression of chLEF1 in chick limb buds 18 and because genetic activation of the Wnt pathway has been observed in most spontaneous colon cancers 1-3. We observed that co-transfection of expression vectors for full length TCF1 or TCF4 and b-catenin with a luciferase reporter gene driven by the LEF1 promoter caused a seven-fold and 4.6-fold activation of luciferase expression respectively (FIG. 4a). Activation was dependent on β-catenin because co-transfection with D19 β-catenin, a mutant that cannot bind to LEF/TCFs (ref. 19), did not allow activation (FIG. 4a). We used DNAase I footprinting and recombinant LEF1 protein to identify two LEF/TCF binding sites at +192 and +283 relative to the LEF1 transcription start site (FIG. 4b). Partially fractionated whole cell extracts from Jurkat T lymphocytes, which express high levels of TCF1, TCF4 and LEF1, protected sequences over the +283 site suggesting that this is a high affinity LEF/TCF binding site. When we deleted this footprinted region (to +262), b-catenin activation of the promoter was reduced from 9.2-fold to 4-fold; when both downstream footprints were deleted, β-catenin activation of the promoter was nearly eliminated (FIG. 4c). Thus, TCF1 or TCF4 together with β-catenin can activate the LEF1 promoter through one or two response elements that lie in an unusual position downstream of the transcription start site. We also observed that b-catenin/TCF complexes can activate the LEF1DN promoter in intron 2, but to a modest level (2-3 fold). Clearly, additional factors or epigenetic mechanisms must modulate the ability of the Wnt pathway to access the LEF1 promoter but not the LEF1DN promoter in colon cancer. To test the model that LEF1 expression is regulated by b-catenin/TCF complexes in colon cancer cells, we co-transfected a GFP/APC (Green Fluorescent Protein/Adenomatous Polyposis Coli) expression plasmid with the LEF1 promoter luciferase reporter construct into SW480 cells (FIG. 4d). This APC fusion protein has previously been shown to reduce b-catenin protein in SW480 cells, and indeed we observed a three-fold decrease in LEF1 promoter activity (FIG. 4d, 1.0 mg GFP/APC). There is no inhibition with the parent GFP expression plasmid but instead a modest increase in luciferase levels (FIG. 4d). We also overexpressed GFP/APC in Colo320 cells, which produce higher detectable levels of LEF1 protein on western blots, and observed a decrease of β-catenin and LEF1 levels, but no detectable decrease of TCF4 protein (FIG. 4d). We conclude that the LEF1 promoter is sensitive to the level of b-catenin in the nucleus of colon cancer cells, and thus is likely to be a Wnt gene target.

Although the current model for colon cancer predicts a correlation between colon tumorigenesis and high levels of LEF/TCF target gene expression, removal of one of these target genes from mice—the Tcfl locus itself—leads to the development of adenomas in the gut and mammary glands5. It has been suggested that loss of Tcf1 reflects loss of the putative tumor suppressor properties of the smaller dominant negative form of TCF1 which must be present in levels that exceed those of full-length TCF1 and TCF4 and therefore TCF1 is a candidate gene for loss of heterozygosity (LOH) in human colon cancer5. However, given our results that the highly similar LEF1 locus has two promoters that are differentially regulated in colon cancer, promoter misregulation at the TCF1 locus is a plausible alternative to TCF1 LOH. The promoter for dominant negative TCF1 could be down-regulated or shut off in cancer and the promoter that drives expression of full-length, β-catenin binding forms could be up-regulated, or turned on. Expression of full length LEF1 and TCF1 in the absence of the moderating influence of their dominant negative isoforms allows for the large pool of β-catenin protein to be fully exploited for target gene activation. LEF1/b-catenin complexes have been shown to transform normal chicken embryo fibroblasts20 but whether this complex contributes to the advancement and/or maintenance of tumors in colon is not known. In addition to providing insight into the mechanism of tumor progression, these genes may be used as important markers of Wntstimulated progression of carcinogenesis.

The following examples are intended to illustrate but not limit the invention.

### EXAMPLE 1

### Methods

In situ hybridization. We performed in situ hybridization of 5 mm sections from paraffm-embedded tissue of normal and malignant colon biopsy samples as described (“Non-radioactive In situ Hybridization”; Roche Molecular Biochemicals) with modifications (T. Milovanovic, T. Truong, and J. L. Marsh). Human TCF4 and LEF1 cDNAs encoding the 3′ untranslated regions were used to generate single-stranded antisense RNA with digitonin-conjugated UTP nucleotides. Probes were hybridized to tissue for 72 hours, then washed and incubated with alkaline phosphatase-conjugated anti-digoxigenin antibody (Roche) for one hour at 37° C. We developed tissues with 5-bromo4-chloro-3-indolylphosphate and 4-nitroblue tetrazolium chloride (BCIP/NBT; Roche), and used a 0.1% Fast Red solution for counterstain. All antisense and sense probes were tested for specificity on human Jurkat T lymphocyte cells which express both LEF1 and TCF4. The sense probes did not produce any detectable signal. Signals were visualized with an Olympus B50 microscope with Nomarski optics and photographs were captured with digital technology within 48 hours of hybridization. Northern Analysis. We analyzed LEF1 expression by Northern analysis of 10 μg of total or 1 μg of polyA+RNA as described previously¹⁰. The LEF1 ORF probe was generated by StyI digestion (nt#821-1894), and the 5′ UTR probe was generated by BgIII digestion (nt#2-761). Melanoma RNA was purified from A2058 cells from a human metastatic melanoma (ATCC#11 147-CRL). Transient Transfection Assays. We subcloned fragments of intron 2 by the indicated enzymes and cloned them in both orientations into the SmaI site of pGL2-Enhancer plasmid (Promega). We transfected 5 μg of each promoter construct with 0.5 μg of CMV-LacZ reporter plasmid into 2017 T lymphocytes. Cell lysates were prepared for luciferase and β-galactosidase assays 20 hours post-transfection¹⁴. To test for dominant negative activity of a truncated LEF1 protein, we co-transfected ΔNLEF1 (aa67-399) with 1 μg of the TOPtk reporter plasmid (gift of Dr. H. Clevers, Univ. Utrecht) and 0.5 μg of CMV-LacZ. To assay for β-catenin regulation of the LEF1 promoter, we co-transfected 2 μg of TCF1 and TCF4 expression plasmids with a luciferase reporter plasmid driven by the LEF1 promoter (B5:−672, +305; ref. 14) and 4 μg of wild type or mutant Δ19 β-catenin expression plasmids into 2017 cells¹⁹. SW480 cells (250,000/35 mm well) were transfected using Effectene (Qiagen; manufacturer's protocols) and 0.5 μg of the B5 LEF1 promoter/luciferase reporter plasmid with 0.1 μg CMVLacZ and the indicated amounts of GFP/APC. Colo320 cells (500,000/35 mm well) were transfected with Effectene and the indicated amounts of GFP/APC expression vector. Whole cells were harvested 24 hours later for western analysis. Western Analysis. We separated proteins from 50,000 Jurkat cells or 75,000 colon cancer cells by SDS-PAGE electrophoresis and probed blots of these gels with the indicated antibodies. TCF1 and TCF4 monoclonal antibodies (Upstate Biotechnology) were used at a 1:1000 dilution to identify cross-reacting polypeptides detected by REMB6 and LEF1 polyclonal antisera. The REMB1 LEF1 monoclonal (Exalpha) was used at a 1:5000 dilution and REMB6 (Exalpha; detects all LEF/TCFs) at a 1:500 dilution. LEF1 polyclonal rabbit antisera (which also detects all LEF/TCF proteins) was used at a 1:1000 dilution. β-catenin levels were analyzed by monoclonal antisera from Transduction Laboratories (1:1000 dilution).

DNAase I Footprinting. Partially purified recombinant LEF1 (10 μg) and Jurkat and HeLa whole cell extracts (50 μg) were used in standard DNAase I footprinting assays as previously described¹⁴. The LEF1 promoter was labeled with ³²P at a phosphatased HindIII site in the polylinker region of B5 plasmid between the promoter and luciferase coding sequences.

Accession Numbers. The nucleotide sequence of the second intronic promoter has been submitted to Genbank (AF288570). Genbank AF288571 lists the nucleotide sequence of the human LEF1 cDNA and amino acid sequence of LEF1.

References:

1. Kinzler, K. & Vogelstein, B. Lessons from hereditary colorectal cancer. Cell 87, 159-170 (1996).

2. Polakis. Wnt signaling and cancer. Genes and Dev. 14, 1837-1851 (2000).

3. Roose, J. & Clevers, H. TCF transcription factors: molecular switches in carcinogenesis. Biochim. Biophys. Acta 1424, M23-37 (1999).

4. Polakis, P., Hart, M. & Rubinfeld, B. Defects in the regulation of beta-catenin in colorectal cancer. Adv. Exp. Med. Biol. 470, 23-32 (1999).

5. Roose, J. et al. Synergy between tumor suppressor APC and the beta-catenin-Tcf4 target Tcf1. Science 285, 1923-1926 (1999).

6. He, T. C. et al. Identification of c-MYC as a target of the APC pathway. Science 281, 1509-1512 (1998).

7. Tetsu, O. & McCormick, F. Beta-catenin regulates expression of cyclin D1 in colon carcinoma cells. Nature 398, 422-426 (1999).

8. Crawford, H. C. et al. The metalloproteinase matrilysin is a target of beta-catenin transactivation in intestinal tumors. Oncogene 18, 2883-2891 (1999).

9. Brabletz, T., Jung, A., Dag, S., Hlubek, F. & Kirchner, T. beta-catenin regulates the expression of the matrix metalloproteinase-7 in human colorectal cancer. Am. J. Pathol. 155, 1033-1038 (1999).

10. Porfiri, E. et al. Induction of a beta-catenin-LEF-1 complex by wnt-1 and transforming mutants of beta-catenin. Oncogene 15, 2833-2839 (1997).

11. Korinek, V. et al. Constitutive transcriptional activation by a beta-catenin-Tcf complex in APC-/- colon carcinoma. Science 275, 1784-1787 (1997).

12. Waterman, M. L., Fischer, W. H. & Jones, K. A. A thymus-specific member of the HMG protein family regulates the human T cell receptor C alpha enhancer. Genes Dev. 5, 656-669 (1991). p0 13. Travis, A., Amsterdam, A., Belanger, C. & Grosschedl, R. LEF-1, a gene encoding a lymphoid-specific protein with an HMG domain, regulates T-cell receptor alpha enhancer function. Genes Dev. 5, 880-894 (1991).

14. Hovanes, K., Li, T. W. H. & Waterman, M. L. The human LEF-1 gene contains a promoter preferentially active in lymphocytes and encodes multiple isoforms derived from alternative splicing. Nucleic Acids Res. 28, 1994-2003 (2000).

15. van de Wetering, M. et al. The human T cell transcription factor-1 gene. Structure, localization, and promoter characterization. J. Biol. Chem. 267, 8530-8536 (1992).

16. Van de Wetering, M., Castrop, J., Korinek, V. & Clevers, H. Extensive alternative splicing and dual promoter usage generate Tcf-protein isoforms with differential transcription control properties. Mol. Cell. Biol. 16, 745-752 (1996).

17. Morin, P. et al. Activation of beta-catenin-Tcf signaling in colon cancer by mutations in beta-catenin or APC. Science 275, 1787-1790 (1997).

18. Kengaku, M. et al. Distinct WNT pathways regulating AER formation and dorsoventral polarity in the chick limb bud. Science 280, 1274-1277 (1998).

19. Prieve, M. G. & Waterman, M. L. Nuclear Localization and formation of b-catenin-Lymphoid Enhancer Factor-1 complexes are not sufficient to activate gene expression. Mol. Cell. Biol. 19, 4503-4515 (1999).

20. Aoki, M., Hecht, A., Kruse, U., Kemler, R. & Vogt, P. K. Nuclear endpoint of Wnt signaling: neoplastic transformation induced by transactivating lymphoid-enhancing factor 1. Proc. Natl. Acad. Sci. U S A 96, 139-144 (1999).

21. Spolski, R. et al. Regulation of expression of T cell gamma chain, L3T4 and Ly-2 messages in Abelson/Moloney virus-transformed T cell lines. Eur. J. Immunol. 18, 295-300 (1988).

22. Carlsson*, P., Waterman*, M. & Jones, K. The hLEF/TCF-1a HMG protein contains a context-dependent transcriptional activation domain that induces the TCRa enhancer in T cells. Genes Dev. 7, 2418-2430 (1993).

Although the invention has been described with reference to the above examples, it will be understood that modifications and variations are encompassed within the spirit and scope of the invention. Accordingly, the invention is limited only by the following claims.

We claim:

1. An isolated polynucleotide comprising a truncated LEF-1 polynucleotide or homolog thereof lacking nucleotides encoding a β-catenin binding domain and not adjacent to nucleotide sequences to which it is naturally adjacent.

2. The polynucleotide of claim 1, wherein the polynucleotide encodes a polypeptide having about 283 amino acids beginning at a methionine codon within exon 3 of the human LEF-1 gene.

3. The polynucleotide of claim 1, wherein the polynucleotide encodes a polypeptide beginning at about amino acid residue 116 of human LEF-1.

4. An isolated polynucleotide having regulatory activity and comprising nucleotides in intron 2 of human LEF-1 gene and within about 50 nucleotides 5′ of the third exon of human LEF-1 gene and homologs thereof.

5. A purified polypeptide encoded by a polynucleotide of claim 1.

6. Isolated antibodies that bind specifically to a polypeptide encoded by a polynucleotide of claim 1, or to immunogenic fragments thereof, with the proviso that the antibodies do not bind to human LEF-1 polypeptide or immunogenic fragments thereof.

7. The antibodies of claim 6, wherein the antibodies are monoclonal.

8. An isolated polynucleotide comprising a polynucleotide sequence of claim 4 operably linked to a polynucleotide of claim 1.

9. A method for diagnosing or monitoring the recurrance or predisposition to colon cancer in a subject comprising detecting the level of expression of full length LEF-1 and truncated LEF-1 polynucleotide or the level of full length LEF-1 and truncated LEF-1 polypeptide in a sample from the subject, wherein an elevated level of full length LEF-1 polynucleotide or polypeptide is indicative of the presence of colon cancer or predisposition thereto.

10. The method of claim 9, wherein the subject is a human.

11. A kit useful for for diagnosing or monitoring the recurrance or predisposition to colon cancer in a subject comprising a first container containing a nucleic acid probe for detecting the level of expression of full length LEF-1 and truncated LEF-1 polynucleotide.

12. A kit useful for for diagnosing or monitoring the recurrance or predisposition to colon cancer in a subject comprising a first container containing an antibody for detecting the level of expression of full length LEF-1 and truncated LEF-1 polypeptide.

13. An isolated polypeptide comprising a LEF1 amino acid sequence consisting of a C-terminal fragment of LEF1, wherein the C-terminal fragment is from amino acid 116 to 398 of LEF1 and wherein the LEF1 amino acid sequence is not adjacent to an amino acid sequence that is naturally adjacent to the LEF1 amino acid sequence.

14. A method of treating or inhibiting colon cancer in a subject comprising contacting a cell with an antagonist of a regulatory region encoding full length LEF-1 or an agonist of the polynucleotide of claim 4, thereby treating or inhibiting colon cancer.

15. A method of treating or inhibiting colon cancer in a subject comprising contacting a cell with an antagonist of a full length LEF-1 polypeptide or an agonist of a truncated LEF-1 polypeptide, thereby treating or inhibiting colon cancer.

16. The method of claim 15, wherein the truncated LEF-1 polypeptide is a polypeptide of claim 5.

17. A method for screening for an agent useful for the treatment of colon cancer comprising contacting a promoter sequence of LEF-1 or a truncated LEF-1 promoter operably associated with a detectable marker with a test agent, and detecting a decrease in detectable marker from the promoter of LEF-1 or an increase in detectable marker from the truncated promoter is indicative of an agent that is useful for the treatment of colon cancer.
